(19) United States
(12) Patent Application Publication
(10) Pub. No.: US 2006/0188418 A1
(43) Pub. Date: Aug. 24, 2006

(54) Multi-functional child care storage

(75) Inventors: Yoon Hee Park, Seoul (KR); Sung Ik Chang, Seoul (KR)

Correspondence Address: BACON & THOMAS, PLLC, 625 SLATERS LANE, FOURTH FLOOR, ALEXANDRIA, VA, 22314, US

(73) Assignee: Daewoo Electronics Corporation, Seoul (KR)

(21) Appl. No.: 11/358,069

(22) Filed: Feb. 22, 2006

(30) Foreign Application Priority Data
Feb. 22, 2005 (KR) 10-2005-0014621
Feb. 22, 2005 (KR) 10-2005-0014622
Mar. 29, 2005 (KR) 10-2005-0025775
May 3, 2005 (KR) 10-2005-0037178

(51) Int. Cl. A61L 2/07

(52) U.S. Cl. 422/292; 422/26

(57) ABSTRACT

A multi-functional child care storage for storing and treating infant care products includes a plurality of functional compartments provided in a main body and having individual spaces divided by a partition member. The functional compartments includes a refrigerating compartment for storing the infant care products at a reduced temperature, a refrigerating and heating compartment for storing, cooling and heating the infant care products, a warming-in-water compartment for warming up the infant care products and a sterilizing compartment for sterilizing the sterilizing and drying. A control unit controls the functional compartments so that each of the functional compartments is maintained at a predetermined temperature.

### FIELD OF THE INVENTION

The present invention relates to a multi-functional child care storage and, more particularly, to a multi-functional child care storage adapted for storing and treating infant food or infant care products within a single unit in various ways on their use and function basis, thus enhancing the convenience in use of infant-related articles and avoiding unnecessary consumption of the time required for child care.

### BACKGROUND OF THE INVENTION

In general, a refrigerator is used to keep food or beverage in a refrigerated or frozen state for a long time or to cool them rapidly. The refrigerator includes a freezer compartment and a refrigerator compartment opened or closed by individual front doors. To supply cool air to the freezer and refrigerator compartments, the refrigerator further includes a compressor, a condenser, a capillary tube and a cooling device to perform a cooling cycle. In the cooling cycle, a compressor compresses a coolant at a high temperature and a high pressure, and provides thus compressed coolant to the condenser. Then, a condenser converts the compressed coolant into a liquid state of a low temperature and a low pressure by releasing heat of the coolant. The low-temperature low-pressure liquid coolant is then converted into a liquid state of a low temperature and a high pressure while it passes through the capillary tube. The low-temperature high-pressure liquid coolant is then directed to a cooling device installed at a rear side of the freezer compartment. The coolant sent to the cooling device is converted into a low-pressure state again while it passes through a coolant pipe of the cooling device, and finally evaporates, thereby reducing the temperature of the freezer compartment and the refrigerator compartment. Such a cooling cycle of the refrigerator is performed repeatedly.

Recently, with a rise in the standard of living, there have been increasing demands for diversified types of special-purpose refrigerators. Developed to meet such needs and commonly used are, for example, a heating cabinet for keeping products warm at a constant temperature, a kimchi refrigerator separately equipped with a heating wire for the ripening of kimchi and an evaporation pipe for the preservation of the ripened kimchi, a cosmetics-exclusive cooler for cooling cosmetics stored in a cosmetics storage compartment by means of a thermoelectric element, and so forth.

Despite the development of such types of special-purpose or multi-purpose refrigerators, no infant care exclusive storage has been developed that increasingly attracts an interest and an investment in recent years.

If infant food or infant care products such as powdered milk, breast milk, liquid milk, weaning food and the like are stored in a conventional household refrigerator, they will be soaked with the odors of other foods stored in the refrigerator. Not only this causes hygienic problems but also result in deterioration in the quality of the stored products. Furthermore, in case of using a microwave oven to warm up the infant care products, e.g., milk or dry milk with water, the warming task should be repeatedly conducted each time such needs arise, which is highly cumbersome and makes it difficult to keep the infant care products at above a prescribed temperature for a long period of time. As an alternative, it would be possible to use a heating cabinet to warm up or preserve milk or dry milk with water. However, such a heating cabinet is usually designed for use at hospitals, drugstores, dining rooms and like places rather than for household use, which means that the heating cabinet is not suitable for baby care. In addition, it costs too much to purchase the heating cabinet for the sole purpose of milk storage.

Moreover, with a view to assure infant hygiene, a general household sterilizing device is often used to sterilize infant care articles such as medical supplies, feeding bottles, gauze handkerchiefs, toys and the like. Inasmuch as the sterilizing device has a design suitable for sterilization of dishware or other utensils used in a kitchen, it is inappropriate for such a sterilizing device to sterilize the infant care articles together with the kitchenware.

Korean Patent Application No. 2004-71011 teaches a multi-functional showcase-type refrigerator incorporating therein a showcase, a sterilizing device, a heating cabinet and a water purifier. This refrigerator is configured to incorporate varying kinds of devices needed in for restaurant business facilities and can be centrally controlled by a microcomputer. Although the refrigerator makes a user conveniently enjoy a variety of functions incorporated therein, the functions and structure thereof are not fit for child care but customized for commercial use with an emphasis placed on the enlargement of its capacity.

In addition to the above, there have been proposed many kinds of sterilizers for sterilizing infant care products, particularly, feeding bottles, one of which is an ultra violet sterilizer described in Korean Patent Application No. 2003-92985. However, most of these sterilizers are merely intended for sterilization of feeding bottles and have no ability to store varying kinds of other infant care products than the feeding bottles on their use basis.

Accordingly, a need has existed for a product capable of storing baby food or infant care products in various ways on their use and function basis. Breaking from the fixed conception that electronic appliances have to be designed and distributed in terms of the housewives' preference or the convenience in an adult life, it is the time to develop new applications and markets of a refrigerator, thus creating a new group of customers.

### SUMMARY OF THE INVENTION

It is, therefore, an object of the present invention to provide a multi-functional child care storage adapted for storing and treating infant food or infant care products within a single unit in various ways on their use and function basis.

In accordance with an aspect of the present invention, there is provided a multi-functional child care storage for storing and treating infant care products, which includes: a main body; a plurality of functional compartments provided in the main body and having individual spaces divided by a partition member, the functional compartments including a refrigerating compartment for storing the infant care products at a reduced temperature, a refrigerating and heating compartment for storing, cooling and heating the infant care products, a warming-in-water compartment for warming up the infant care products and a sterilizing compartment for sterilizing the sterilizing and drying; and a control unit for controlling the functional compartments so that each of the functional compartments is maintained at a predetermined temperature.

### BRIEF DESCRIPTION OF THE DRAWINGS

The above and other objects and features of the present invention will become apparent from the following description of preferred embodiments given in conjunction with the accompanying drawings, in which:

FIG. 1 is a perspective view of a multi-functional child care storage in accordance with the present invention;

FIG. 2 is a perspective view showing the multi-functional child care storage in accordance with the present invention, with doors thereof opened;

FIG. 3 depicts a cross-sectional view showing a refrigerating compartment of the multi-functional child care storage in accordance with the present invention;

FIG. 4 represents a cross-sectional view showing a modified refrigerating compartment of the multi-functional child care storage in accordance with the present;

FIG. 5 is a side cross-sectional view showing a refrigerating and heating compartment of a first embodiment employed in the multi-functional child care storage according to the present invention;

FIG. 6 is a side cross-sectional view showing a refrigerating and heating compartment of a second embodiment employed in the multi-functional child care storage according to the present invention;

FIG. 7 is a side cross-sectional view showing a refrigerating and heating compartment of a third embodiment employed in the multi-functional child care storage according to the present invention;

FIG. 8 illustrates a front cross-sectional view of a sterilizing compartment and a warming-in-water compartment in the multi-functional child care storage according to the present invention; and

FIG. 9 shows a block diagram of a circuit for controlling the multi-functional child care storage in accordance with the present invention.

### DETAILED DESCRIPTION OF THE PREFERRED EMBODIMENTS

Hereinafter, preferred embodiments of the present invention will be described in detail with reference to the accompanying drawings, in such a manner that those skilled in the art can readily implement the present invention.

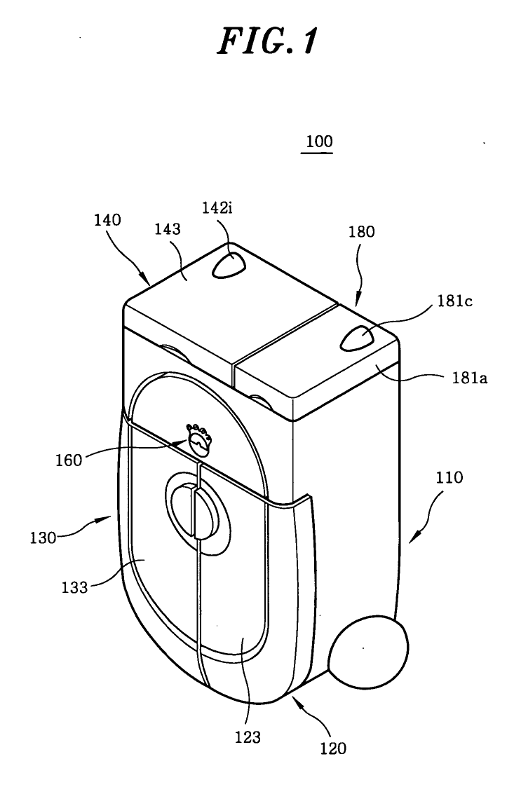

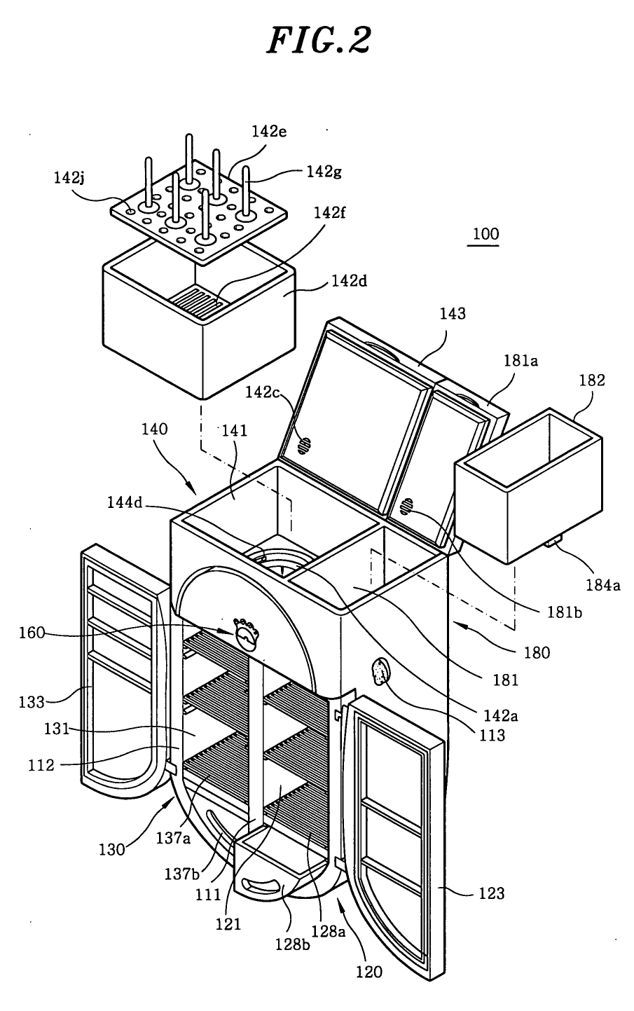

FIG. 1 is a perspective view of a multi-functional child care storage in accordance with the present invention, and FIG. 2 is a perspective view showing an in-use state of the multi-functional child care storage in accordance with the present invention.

As shown in the figures, a multi-functional child care storage 100 includes a main body 110 whose internal space is divided by a partition member in horizontal direction into an upper body and a lower body. The lower body is further divided by a partition member 111 in vertical direction into a refrigerating compartment 120 and a refrigerating and heating compartment 130, while the upper body is further divided by the partition member 111 into a sterilizing compartment 140 and a warming-in-water compartment 180, thus creating a plurality of functional compartments. In each of the upper and lower bodies, an insulating material 113 for thermal insulation is filled in a wall member 112 and the partition member 111. Although the upper and lower bodies have two compartments as shown and disclosed above, it is possible to allow the upper and lower bodies to have only one functional compartment. For example, the upper body (or the lower body) may be made exclusively for a refrigerating compartment or a refrigerating/heating compartment (or a sterilizing compartment or a warming-in-water compartment).

Provided on the outer front surface of the upper body is a control unit 160 that includes switches and buttons for controlling operations of the refrigerating compartment 120, the refrigerating and heating compartment 130, the sterilizing compartment 140, the warming-in-water compartment 180, and so forth.

The lower body is provided with a first door 123 for opening and closing the refrigerating compartment 120 and a second door 133 for opening and closing the refrigerating and heating compartment 130. Likewise, the upper body is provided with a third door 143 for opening and closing the sterilizing compartment 140 and a fourth door 181a for opening and closing the warming-in-water compartment 180.

A first thermoelectric element (not shown in FIGS. 1 and 2) is provided in the refrigerating compartment 120 to cool down a refrigerating space 121. Racks 128a and a storage drawer 128b are detachably installed within the refrigerating space 121.

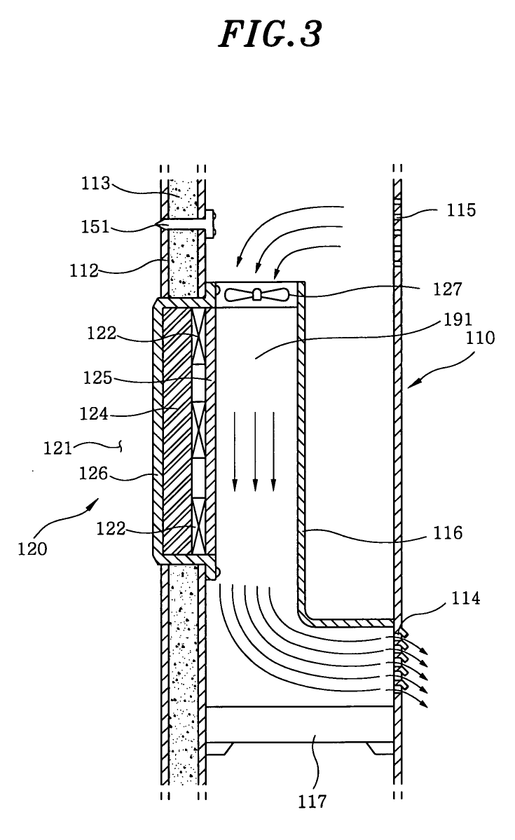

Referring to FIG. 3, the detailed structure of the refrigerating compartment 120 is illustrated in a cross-sectional view.

At the rear side of the refrigerating space 121, the refrigerating compartment 120 has an air-circulated cooling room 191 providing an air circulation route through which the ambient air is introduced and discharged to the outside to dissipate the heat emitted from a heat radiation plate 125 of a first thermoelectric element 122. The heat radiation plate 125 of the first thermoelectric element 122 protrudes into the air-circulated cooling room 191. A first heat radiation fan 127 is installed at the heat emitting side of the first thermoelectric element 122, i.e., at the side of the heat radiation plate 125 to dissipate the heat of the first thermoelectric element 122 to the outside through a heat radiation outlet 114 of the main body 110. Also, the main body 110 has an air inlet 115 through which the atmospheric air is drawn toward the first heat radiation fan 127.

The first thermoelectric element 122 is installed on the wall member 112 lying at the back side of the refrigerating space 121, the number of which may be one or plural. Responsive to the supply of an electric current, the first thermoelectric element 122 absorbs heat at one side and emits the heat thus absorbed at the other side. The first thermoelectric element 122 has a heat absorbing plate 124 provided at the side of the refrigerating space 121 for the purpose of cooling the latter. The heat radiation plate 125 is placed at the opposite side from the heat absorbing plate 124. Thus, the first thermoelectric element 122 emits coldness into the refrigerating space 121 through the heat absorbing plate 124 and radiates the heat to the opposite side of the refrigerating space 121 through the heat radiation plate 125. A coldness diffusion plate 126 made of a high conductivity material, e.g., aluminum, is provided at the side of the heat absorbing plate 124 and exposed to the refrigerating space 121 so that the coldness emitted from the first thermoelectric element 122 can be rapidly diffused to each and every part of the refrigerating space 121.

Furthermore, in the refrigerating compartment 120, an evaporation dish 117 for collecting and evaporating condensed water is disposed underneath the heat radiation plate 125 of the first thermoelectric element 122. Also collected in the evaporation dish 117 is the condensed water drained from the refrigerating and heating space 131 of the refrigerating and heating compartment 130 through a drainage line 118. The condensed water collected in the evaporation dish 117 is rapidly evaporated by the heat radiated from the heat radiation plate 125 of the first thermoelectric element 122 and supplied by the blowing action of the first heat radiation fan 127.

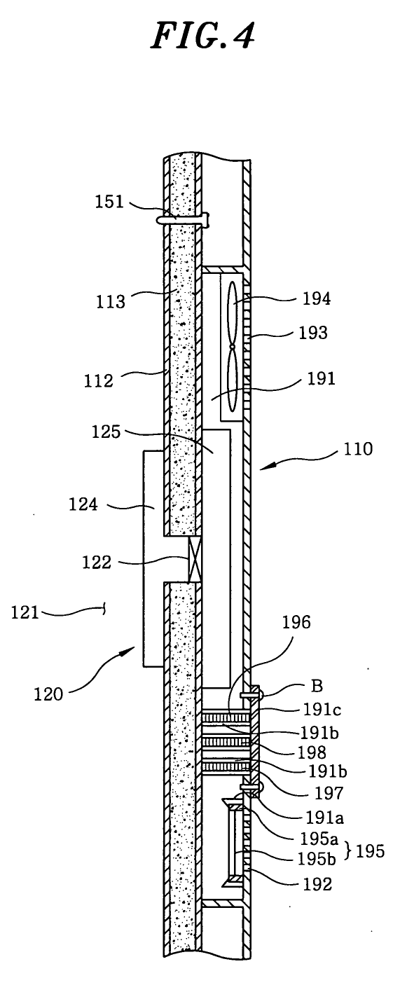

FIG. 4 is a cross-sectional view showing the detailed structure of a refrigerating compartment, which is a modified (second) embodiment of the refrigerating compartment 120 illustrated in FIG. 3. Like parts or components will be designated with the same reference numerals as in FIG. 3 and will be given no description in that regard.

Just like in the embodiment shown in FIG. 3, an air-circulated cooling room 191 is provided at the rear side of a refrigerating space 121.

In the air-circulated cooling room 191, there are provided an inlet 192, an outlet 193, and a cooling fan 194 installed in the vicinity of the outlet 193. Serially provided inside the air-circulated cooling room 191 are a pre-filter 195 and an end filter 196, each of which serves to purify the air discharged from the air-circulated cooling room 191.

The pre-filter 195 is detachably attached to the side of the inlet 192 in the air-circulated cooling room 191 by means of a hook 191a. The pre-filter 195 has a frame 195a forming a rim thereof, inside of which a reticular structure member or a non-woven fabric 195b is fixedly secured in order to remove relatively large-sized dusts in the air suctioned through the inlet 192.

The end filter 196 removes fine dusts existing in the air which has passed the pre-filter 195, ensuring that the air is finally exhausted through the outlet 193 in a clean state. Preferred examples of the end filter 196 include a HEPA (High Efficiency Particulate Air) filter. The HEPA filter is made of an electrically charged special fiber material and exhibits a strong adsorption force great enough to remove acarids, molds and viruses as well as the fine dusts contained in the air.

Disposed between the pre-filter 195 and the end filter 196 are a nano silver filter 197 containing extremely fine silver particles and a nano carbon filter 198 mainly composed of carbon particles. Unlike the illustrated embodiment, it would be possible to employ only one of the nano silver filter 197 and the nano carbon filter 198. The nano silver filter 197 is made of a non-woven fabric or a filter paper containing extremely fine silver particles and is excellent in its antibacterial, deodorization and sterilization ability. The nano carbon filter 198 is a charcoal powder filter for deodorization and has a strong adsorbability great enough to remove offensive odors, tobacco smoke, and other toxic gases, thus making the air clean and fresh.

The end filter 196, the nano silver filter 197 and the nano carbon filter 198 are slidably fitted and detachably coupled at their lateral edges to a plurality of fixing ribs 191b formed in the air-circulated cooling room 191. A cover 199 is secured to one side of the air-circulated cooling room 191 by means of a bolt B so that, when the cover 199 is removed, it becomes possible to gain access to the end filter 196, the nano silver filter 197 and the nano carbon filter 198 for the purpose of cleansing and replacement.

Referring again to FIG. 1, at the front side of the main body, the refrigerating and heating compartment 130 is provided with a second door 133 for opening and closing the refrigerating and heating space 131. Racks 137a and a storage drawer 137b are detachably installed within the refrigerating and heating space 131.

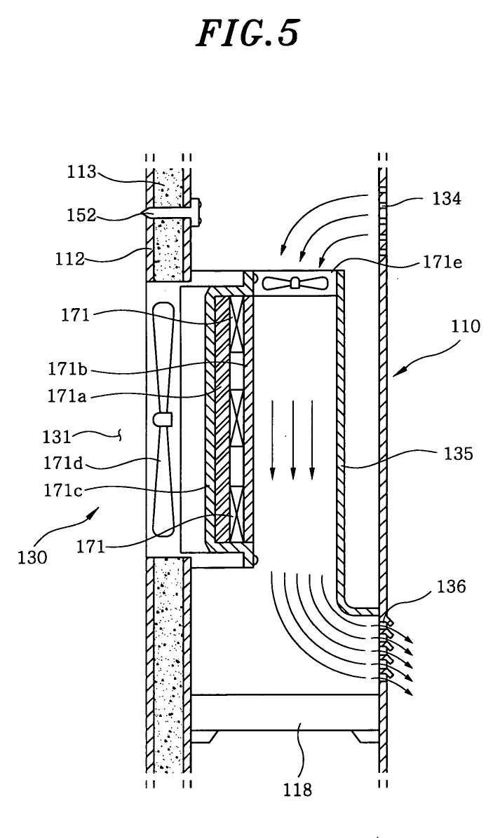

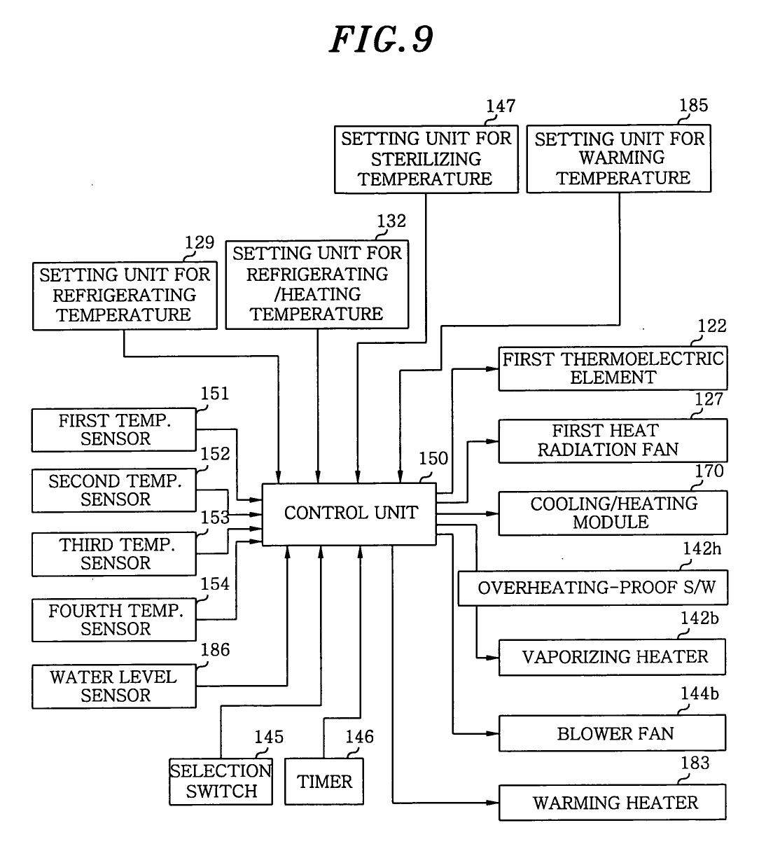

Referring to FIG. 5, the detailed structure of the refrigerating and heating compartment 130 of the multi-functional storage according to the present invention is illustrated in a cross-sectional view. As illustrated, the refrigerating and heating compartment 130 is maintained at a predetermined temperature by means of a cooling and heating module 170 (see, FIG. 9). The cooling and heating module 170 includes a second thermoelectric element 171 that serves to cool down or heat up the refrigerating and heating space 131 depending on the direction of an electric current applied thereto and also functions to control the heat absorption amount and the heat emission amount depending on the intensity of the electric current. Radiation plates 171a and 171b for emitting coldness and heat are respectively installed at opposite sides of the second thermoelectric element 171. A diffusion plate 171c that serves to rapidly and extensively dissipate the coldness and the heat is provided to face the refrigerating and heating space 131. A diffusion fan 171d and a second radiation fan 171e are disposed at the opposite sides of the second thermoelectric element 171.

The diffusion fan 171d is adapted to rapidly dissipate the coldness or the heat emitted from the radiation plate 171a into the refrigerating and heating space 131. During the time when the second thermoelectric element 171 cools down the refrigerating and heating space 131, the second radiation fan 171e discharges the heat emitted from the radiation plate 171b and the air introduced through an inlet 134 formed on the main body 110 at the rear side of the refrigerating and heating space 131, along a duct 135 and then through a heat radiation outlet 136 formed on the main body 110.

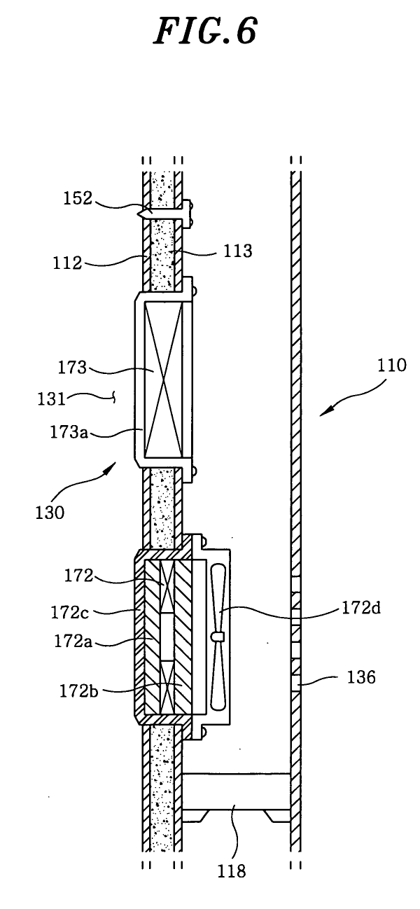

FIG. 6 illustrates a second modified embodiment of the refrigerating and heating compartment shown in FIG. 5. In the second modified embodiment, the cooling and heating module 170 includes a third thermoelectric element 172 and a first heater 173. As in the first modified embodiment, a heat absorbing plate 172a and a heat emitting plate 172b is respectively provided on the opposite sides of the third thermoelectric element 172. A coldness diffusion plate 172c is installed in contact with the heat absorbing plate 172a and a third heat radiation fan 172d is provided in alignment with the heat emitting plate 172b to discharge the heat through a heat radiation outlet 136. The first heater 173 is arranged in the inner side of a refrigerating and heating space 131. A heating block with a heating wire for generating heat in response to the supply of an electric current may be used as the first heater 173. A heat diffusion plate 173a made of a high conductivity material, e.g., aluminum, is coupled to the first heater 173 at the side of the refrigerating and heating space 131. The heat diffusion plate 173a is exposed to the refrigerating and heating space 131 and serves to rapidly dissipate the heat generated from the first heater 173.

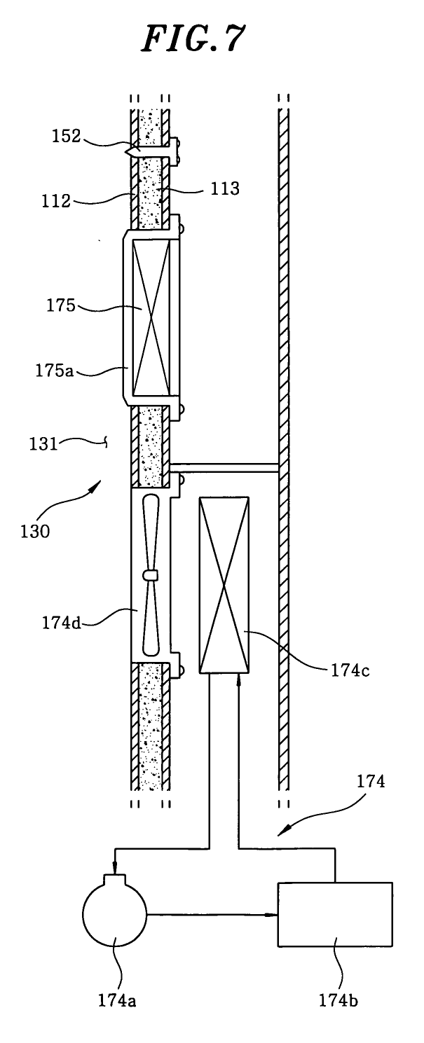

FIG. 7 illustrates a third modified embodiment of the refrigerating and heating compartment shown in FIG. 5. In the third modified embodiment, the cooling and heating module 170 includes a cooler 174 absorbing the ambient heat through the use of a circulating coolant and a second heater 175. The cooler 174 includes a compressor 174a, a condenser 174b and an evaporator 174c, and is adapted to supply coldness to a refrigerating and heating space 131 by performing a cooling cycle comprised of a series of processes, i.e., compressing, condensing and evaporating. In the cooling cycle, a coolant of high temperature and high pressure compressed by the compressor 174a is fed to the condenser 174b which in turn condenses the coolant into a state of low temperature and low pressure, thus causing the coolant to release heat. Then, the condensed coolant passes through the evaporator 174c and absorbs the ambient heat to eventually generate coldness. The coldness thus generated is supplied to the refrigerating and heating space 131 by the diffusion fan 174d. Such a cooling cycle is repeatedly conducted to cool down and circulate the coolant. As with the first heater 173 of the second modified embodiment, the second heater 175 is provided with a heat diffusion plate 175a for rapidly dissipating heat into the refrigerating and heating space 131.

Referring again to FIG. 1, the sterilizing compartment 140 has a third door 143 attached to the upper body for opening and closing the sterilizing space 141.

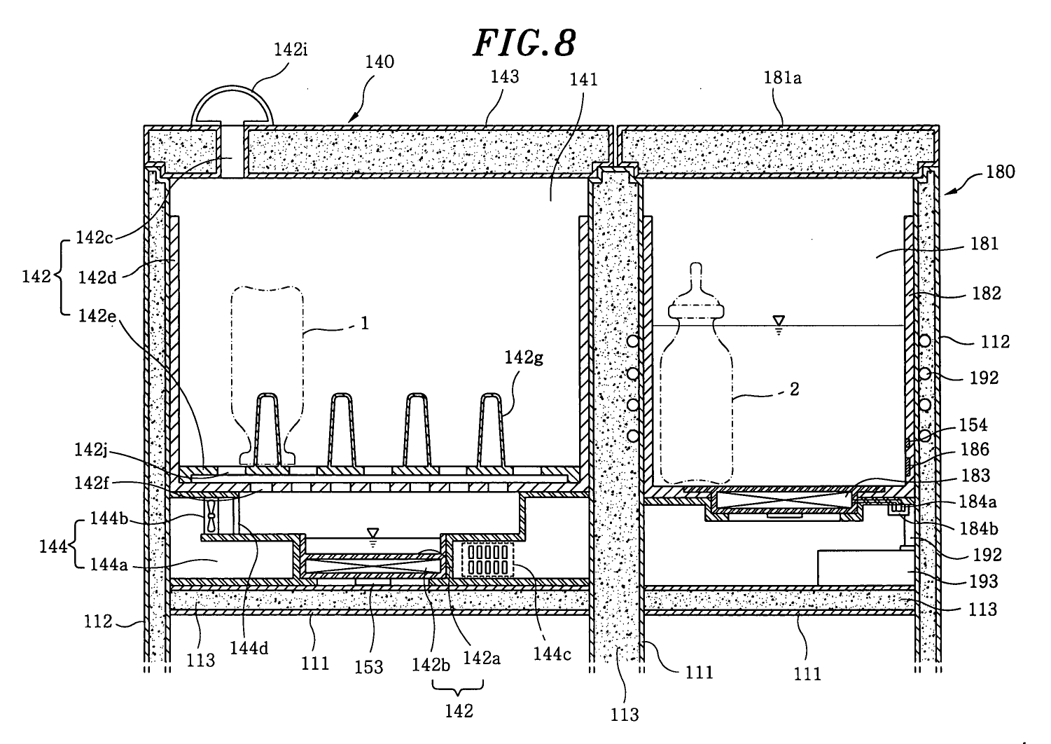

As illustrated in FIG. 8, provided inside the sterilizing compartment 140 is a steam sterilizing unit 142 for sterilizing the sterilizing space 141 with steam. The steam sterilizing unit 142 includes a vaporizing vessel 142a made of metallic material such as stainless steel and placed on the bottom surface of the sterilizing space 141. The vaporizing vessel 142a is filled with water. The steam sterilizing unit 142 further includes a vaporizing heater 142b installed at the bottom of the vaporizing vessel 142a for heating the vaporizing vessel 142a to thereby vaporize the water contained therein. An overheating-proof switch 142h, e.g., a bimetal switch, is arranged in the vaporizing vessel 142a to cut off the current supply at the time when the vaporizing heater 142b is heated beyond a predetermined temperature.

Further, the sterilizing compartment 140 is further provided with a steam vent hole 142c formed in the third door 143 for exhausting the steam from the sterilizing space 141 and supporting stands 142d and 142e for retaining an infant care product 1 to be sterilized within the vaporizing vessel 142a. In order to assure that a user is kept from contacting the steam, a cover 142i for closing the steam vent hole 142c is installed on the top surface of the third door 133. The cover 142i allows the steam exhausted through the steam vent hole 142c to be directed to the backside of the main body 110. Within the sterilizing compartment 140, a sterilizing basket 142d is disposed in the sterilizing space 141 to for keeping the infant care product 1 spaced apart from the vaporizing vessel 142a and helping a user to take out the infant care product 1 from the sterilizing space 141 at the end of sterilization. Also disposed in the sterilizing compartment 140 is a sterilizing base 142e having a plurality of steam passage holes 142j and a plurality of posts 142g for holding the infant care product 1 such as a feeding bottle.

Moreover, a drying unit 144 is provided within the sterilizing compartment 140, which serves to dry the infant care product 1 sterilized in the sterilizing space 141. The drying unit 144 includes a suction duct 144a through which the air is introduced into the sterilizing space 141 from the outside and a blower fan 144b mounted on the suction duct 144a for blowing the air into the sterilizing space 141. At the inlet side of the suction duct 144a, a filter 144c is removably attached to the main body 110 for removing harmful bacteria, fine dusts and other foreign matters borne by the air introduced into the suction duct 144a from the outside. The suction duct 144a is configured in such a manner that an outlet side end 144d thereof lies in contiguity with the vaporizing vessel 142a of the steam sterilizing unit 142. Thus, the heat generated by the vaporizing heater 142b applied to the vaporizing vessel 142a is fed into the sterilizing space 141 together with the air blown by the blower fan 144b, thereby shortening the drying time.

As will be discussed with reference to FIG. 9, the sterilizing compartment 140 further includes a selection switch 145 manipulated by a user to select an independent operation of one of the steam sterilizing unit 142 (vaporizing heater 142b) and the drying unit 144 (blower fan 144b) and a successive operation of both of the steam sterilizing unit 142 and the drying unit 144, and a timer 146 for setting the operating time of the steam sterilizing unit 142 or the drying unit 144. The timer 146 may be operatively associated with the selection switch 145 so that the timer 146 can set the operating time of the steam sterilizing unit 142 or the drying unit 144, whichever is selected by the selection switch 145.

Turning back to FIGS. 1 and 8, the warming-in-water compartment 180 is used to warm up breast milk or powdered milk filled in a feeding bottle and the like. It would be preferred that the sterilizing compartment 140 and the warming-in-water compartment 180 have a smaller capacity than the refrigerating compartment 120 or the refrigerating and heating compartment 130.

The warming-in-water compartment 180 includes a warming vessel 182 separably mounted in a warming space 181 and filled with water, and a warming heater 183 for warming up the water filled in the warming vessel 182.

The warming heater 183 is constructed from a heating block with internally disposed heating wires and directly attached to the bottom of the warming vessel 182. Alternatively, the warming heater 183 may be installed in the warming space 181 such that it can be separated from the warming vessel 182. Power supply connectors 184a and 184b are provided to supply an electric current to the warming heater 183 when the warming vessel 182 is placed in the warming space 181. The power supply connectors 184a and 184b are in the form of a separably coupled receptacle and plug. The power supply connectors 184a and 184b are separated from each other in the event that the warming vessel 182 is removed from the warming space 181, and coupled together when the warming vessel 182 is placed into the warming space 181, thus supplying an electric current to the warming heater 183 in a stable manner.

Referring now to FIG. 9, there is shown a block diagram of a circuit for controlling the multi-functional child care storage in accordance with the present invention.

A control unit 150 is adapted to control the operation of the first thermoelectric element 122 and the first heat radiation fan 127 provided in the refrigerating compartment 120, the operation of the cooling and heating module 170 disposed in the refrigerating and heating compartment 130, the operation of the steam sterilizing unit 142 and the drying unit 144 arranged in the sterilizing compartment 140, and the operation of the warming heater 183 provided in the warming-in-water compartment 180.

Responsive to the temperature signals issued from first and second temperature sensors 151 and 152 respectively disposed in the refrigerating space 121 and the refrigerating and heating space 131, the control unit 150 controls the first thermoelectric element 122 of the refrigerating compartment 120 and the cooling and heating module 170 of the refrigerating and heating compartment 130 in such a manner that the refrigerating compartment 120 and the refrigerating and heating compartment 130 can be kept at the temperature stored in a setting unit for refrigerating temperature 129 and a setting unit for refrigerating/heating temperature 132.

Further, in response to the temperature signals generated from third and fourth temperature sensors 153 and 154 respectively disposed in the sterilizing space 141 and the warming space 181, the control unit 150 controls the vaporizing heater 142b and the warming heater 183 in such a manner that the sterilizing compartment 140 and the warming-in-water compartment 180 can be maintained within the temperature stored in a setting unit for sterilizing temperature 147 and a setting unit for warming temperature 185.

The setting unit 185 is utilized in setting a warming temperature at which the feeding bottle 2 or the like is warmed up in the warming-in-water compartment 180. The control unit 150 controls the warming heater 183 to maintain the temperature set in the setting unit 185. A water level sensor 186 is attached to the warming vessel 182 for detecting the water level within the warming vessel 182 to avoid such a situation that the warming vessel 182 is overheated by the warming heater 183. Detection signals outputted from the water level sensor 186 are sent to the control unit 150 which in turn stops the operation of the warming heater 183 if the water level within the warming vessel 182 is equal to or below a predetermined water level.

The fourth temperature sensor 154 is installed in the warming vessel 182 to accurately detect the temperature during the process of warming. The fourth temperature sensor 154, the water level sensor 186 and the warming heater 183 are supplied with an electric current through the power supply connectors 184a and 184b.

Description will now be given regarding the operation and action of the multi-functional child care storage set forth above.

Infant care food or products, particularly, milk, breast milk, medical supplies for infant, cosmetics for infant are stored in the refrigerating compartment 120. If an electric current is applied to the first thermoelectric element 122 of the refrigerating compartment 120, coldness is emitted from the first thermoelectric element 122 and dissipated into the refrigerating space 121 through the heat absorbing plate 124 and the coldness diffusion plate 126, thus cooling down the refrigerating space 121. At this time, the heat radiated from the first thermoelectric element 122 is transferred to the heat radiation plate 125 and discharged through the heat radiation outlet 114 of the main body 110 by the blowing action of the first heat radiation fan 127.

The temperature within the refrigerating compartment 120 is controlled by the control unit 150 in proportion to the intensity of the electric current supplied to the first thermoelectric element 122. In other words, the control unit 150 controls the temperature in the refrigerating space 121 in response to the detection signals from the first temperature sensor 151 so that the refrigerating space temperature can be substantially equal to the temperature inputted in the setting unit 129.

Responsive to the detection signals issued from the second temperature sensor 152 disposed in the refrigerating and heating space 131, the control unit 150 controls the cooling and heating module 170 so that the refrigerating and heating compartment 130 can be kept at the temperature set in the setting unit 132. The temperature in the refrigerating and heating space 131 may be, for example, 3-4° C. for weaning vegetables or fruits, 7° C. for infant cosmetics, 10° C. for infant medical supplies, 8-12° C. for tropical fruits, and 35-40° C. for hot beverage or herb medicines. It goes without saying that the temperature in the refrigerating and heating space 131 may be arbitrarily changed depending on the kinds of the food and products to be stored.

Feeding bottles, gauze handkerchiefs, toys and the like are stored in the sterilizing space 141 of the sterilizing compartment 140. These products are sterilized using the steam generated by vaporizing the water filled in the vaporizing vessel 142a of the steam sterilizing unit 142. At the end of the sterilizing process, the infant care products are dried by the air fed from the blower fan 144b of the drying unit 144. In the meantime, the air introduced into the sterilizing space 141 by the blower fan 144b is the clean one that has passed the filter 144c to remove harmful bacteria or other alien matters. The air flows along the suction duct 144a and is sprayed at a position adjacent to the vaporizing vessel 142a. This makes it possible to take advantage of the heat generated from the vaporizing heater 142b and applied to the vaporizing vessel 142a, thus shortening the time required for drying the infant care products such as feeding bottles and the like.

Use of the selection switch 145 makes it possible either to operate one of the steam sterilizing unit 142 and the drying unit 144 or to operate both of the steam sterilizing unit 142 and the drying unit 144 one after another. The operating time of these means 142 and 144 can be properly set through the use of the timer 146. Examples of the functions selectable by manipulating the selection switch 145 include a steam sterilizing function using the vaporizing heater 142b, a drying function using the blower fan 144b, a sterilizing and drying function using the vaporizing heater 142b and the blower fan 144b in combination, and a sterilizing, drying and preserving function. According to the sterilizing, drying and preserving function, a preserving operation is automatically conducted after the sterilizing and drying operations has been finished. The preserving operation is to repeatedly dry the stored infant care products at a predetermined time interval (e.g., 30 minutes) for a short period of time (e.g., 5 minutes). This helps prevent recontamination of the infant care products by residual moisture, particularly when the sterilized and dried products are stored for a long period of time without any use.

The warming-in-water compartment 180 employs a warming system wherein a prescribed amount of water is heated up to above 80° C. to thereby raise the temperature of, e.g., a feeding bottle. In addition, use of the setting unit 185 allows a user to select the warming temperature at his or her desire, which makes it possible to rapidly heat the in-bottle water up to a temperature of about 50° C. suitable for mixing powdered milk or a temperature of 37-40° C. suitable for feeding milk. Thus, the warming-in-water compartment 180 can be controlled like a bottle warmer. The control unit 150 recognizes the temperature within the warming vessel 182 based on the detection signals from the fourth temperature sensor 154 and controls the warming heater 183 such that the temperature within the warming vessel 182 remains substantially equal to the temperature set in the setting unit 185. Examples of the bottle warmer functions selectable by manipulating the setting unit 185 include a mode-based warming function and a temperature preserving function, as well as a time-based warming function.

The mode-based warming function may be divided into a feeding bottle mode, a pack mode, a slow mode (thawing mode or night mode). The feeding bottle mode is to warm up a general feeding bottle to a temperature of about 70° C. The pack mode is to warm up a packed product to a relatively low temperature, e.g., 50° C. Unlike the above two modes that can be completed within about 10 minutes, the slow mode (thawing mode or night mode) is to warm up a product step by step over a relatively long period of time (e.g., 3 hours) and then maintain the temperature for an extended period of time (e.g., 3 hours). In the slow mode, it is possible to slowly thaw frozen breast milk with a minimized likelihood of destroying its nutrients and preserve the milk for a prolonged period of time in preparation for nighttime feeding of the same.

In the meantime, the temperature preserving function can be set independently or in combination with the mode-based warming function. The temperature preserving function is automatically initiated just after the warming function of the feeding bottle mode or the pack mode has come to an end, or may be independently set to preserve a hot product at a proper temperature. Such a temperature preserving function is automatically terminated as soon as the sum of the warming time and the preserving time reaches a preset time (e.g., 6 hours).

Thank to the fact that the infant care products can be kept at an elevated temperature by use of the temperature preserving function, it becomes possible to eliminate the need to reheat the products later. Furthermore, use of the warming function in the slow mode (thawing mode or night mode) makes it possible to warm up and preserve infant care products such as breast milk and the like without destroying their nutrients.

As described in the foregoing, according to the present invention, it is possible to store infant food or infant care products within a single unit in various ways on their use and function basis. This increases the convenience in use of infant care products, allows a user to efficiently utilize a household infant care space, and prevents unnecessary waste of the time required for infant care. Provision of a warming-in-water compartment enables a user to conveniently warm up breast milk, powdered milk and water, liquid herb medicines and so forth with minimized possibility of destroying nutrients, which helps an infant to take nutrition. Furthermore, provision of a refrigerating and heating compartment that permits conversion between a refrigerating function and a heating function and allows a user to arbitrarily control the temperature thereof can enhance the storage efficiency of infant care products. In addition, provision of a sterilizing compartment that sterilizes infant care products with steam makes it possible to conveniently and hygienically sterilize the infant care products.

While the invention has been shown and described with respect to the preferred embodiments, it will be understood by those skilled in the art that various changes and modifications may be made without departing from the spirit and scope of the invention as defined in the following claims.

What is claimed is:

1. A multi-functional child care storage for storing and treating infant care products, comprising:
a main body;
a plurality of functional compartments provided in the main body and having individual spaces divided by a partition member, the functional compartments including a refrigerating compartment for storing the infant care products at a reduced temperature, a refrigerating and heating compartment for storing, cooling and heating the infant care products, a warming-in-water compartment for warming up the infant care products and a sterilizing compartment for sterilizing the sterilizing and drying; and
a control unit for controlling the functional compartments so that each of the functional compartments is maintained at a predetermined temperature.

2. The multi-functional child care storage of claim 1, wherein the refrigerating compartment includes:
a door for opening and closing a refrigerating space defined by the partition member; and
a thermoelectric element for cooling down the infant care products stored in the refrigerating space to the predetermined temperature.

3. The multi-functional child care storage of claim 1, wherein the refrigerating and heating compartment includes:
a door for opening and closing a refrigerating and heating space defined by the partition member; and
a thermoelectric element for cooling down or heating up the infant care products stored in the refrigerating and heating space to the predetermined temperature.

4. The multi-functional child care storage of claim 1, wherein the refrigerating and heating compartment includes:
a door for opening and closing a refrigerating and heating space defined by the partition member;
a thermoelectric element for cooling down the infant care products stored in the refrigerating and heating space to the predetermined temperature; and
a heater for heating up the infant care products stored in the refrigerating and heating space to the predetermined temperature.

5. The multi-functional child care storage of claim 1, wherein the refrigerating and heating compartment includes:
a door for opening and closing a refrigerating and heating space defined by the partition member;
a cooler for circulating a coolant to absorb ambient heat to maintain the infant care products stored in the refrigerating and heating space at the predetermined temperature; and
a heater for heating up the infant care products stored in the refrigerating and heating space to the predetermined temperature.

6. The multi-functional child care storage of claim 1, wherein the sterilizing compartment includes:
a door for opening and closing a sterilizing space defined by the partition member;
a vaporizing vessel provided within the sterilizing space and filled with water;
a vaporizing heater for heating the vaporizing vessel to vaporize the water into steam and dispersing the steam in the sterilizing space; and
a steam vent hole for exhausting the steam from the sterilizing space to the outside.

7. The multi-functional child care storage of claim 6, wherein the sterilizing compartment further includes:
an overheating-proof switch for cutting off power supply when the sterilizing space is heated up above a predetermined temperature; and
a supporting stand provided above the vaporizing vessel for supporting the infant care products to be sterilized.

8. The multi-functional child care storage of claim 6, wherein the sterilizing compartment further includes a drying means for drying the infant care products after the infant care products have been sterilized in the sterilizing space, the drying means comprises:
a suction duct through which an air is introduced into the sterilizing space;
a blower fan for blowing the air toward the sterilizing space through the suction duct; and
a filter for removing foreign matters or harmful bacteria from the air passing through the suction duct
wherein the suction duct has an outlet end disposed in contiguity with the vaporizing vessel.

9. The multi-functional child care storage of claim 8, wherein the sterilizing compartment further includes:
a selection switch for selecting an independent operation of one of the vaporizing heater and the blower fan and a successive operation of the vaporizing heater and the blower fan; and
a timer for setting an operating time of the vaporizing heater or the blower fan.

10. The multi-functional child care storage of claim 1, wherein the warming-in-water compartment includes:
a door for opening and closing a warming space defined by the partition member;
a warming vessel separably placed within the warming space and filled with water;
a warming heater for warming up the water filled in the warming vessel; and
a warming temperature setting part for setting a temperature in the warming space.

11. The multi-functional child care storage of claim 10, wherein the warming-in-water compartment further includes:
a water level sensor provided in the warming vessel for sensing a water level of the warming vessel to issue detection signals,
wherein the control unit is adapted to, in response to the detection signals, stop an operation of the warming heater if the water level in the warming vessel is below a predetermined level.
